(19) United States
(12) Patent Application Publication
(10) Pub. No.: US 2011/0281947 A1
(43) Pub. Date: Nov. 17, 2011

(54) METHODS OF ADMINISTERING DICLOFENAC COMPOSITIONS FOR TREATING PHOTODAMAGED SKIN

(76) Inventors: Michael T. Nordsiek, Berwyn, PA (US); Brent Cowgill, Collegeville, PA (US); James Ciciriello, Downingtown, PA (US)

(21) Appl. No.: 13/193,142

(22) Filed: Jul. 28, 2011

Related U.S. Application Data
(62) Division of application No. 12/081,488, filed on Apr. 16, 2008, now abandoned.
Application No. 12/081,488 is a continuation of application No. 10/752,148, filed on Jan. 6, 2004, now abandoned.

(51) Int. Cl. A61K 31/196; A61P 17/00

(52) U.S. Cl. 514/567

(57) ABSTRACT

A method of treating at least one dermatological disorder selected from the group consisting of photodamage-induced wrinkles, rosacea, and acne, includes: (a) providing a composition containing 1-3 wt. % diclofenac or a salt thereof, 0.5-1.5 wt. % benzyl alcohol; 10-30 wt. % PEG monomethyl ether, 0.1-3.0 wt. % sodium hyaluronate; and 70-80 wt. % water; and (b) applying the composition to skin of a person in need of the treating in an amount effective to treat the at least one dermatological disorder.

### BACKGROUND OF THE INVENTION

1. Field of Invention

This invention relates to compositions comprising diclofenac, and more specifically to such compositions for topical administration.

2. Description of Related Art

Ortho-((2,6-dichlorophenyl)amino)phenylacetic acid (hereinafter referred to as diclofenac) and its pharmaceutically acceptable salts are known compounds useful in the treatment of inflammation. A number of patents disclose compositions containing diclofenac, which are for topical administration.

U.S. Pat. No. 6,387,383 to Dow et al. discloses low viscosity topical compositions useful for treating an inflammatory skin disorder, acne, or rosacea. Diclofenac is disclosed and claimed as an active ingredient.

U.S. Pat. No. 6,193,996 to Effing et al. discloses a pressure sensitive skin adhesive comprising: (a) a copolymer of one or more alkyl(meth)acrylates containing 4 to 12 carbon atoms in the alkyl group and one or more hydrophilic monomers; (b) a mixture of penetration enhancers comprising: (i) an alkyl ester of an aliphatic monocarboxylic acid containing 1 to 5 carbon atoms in the alkyl group (ii) an alkyl pyrrolidone and (iii) an alkane polyol; and (c) a therapeutically effective amount of diclofenac or a pharmaceutically acceptable salt thereof.

U.S. Pat. No. 5,895,649 to De Lacharriere et al. discloses topically applicable pharmaceutical/dermatological/cosmetic compositions, well suited for the therapeutic treatment of neurogenic red skin blotches and rosacea afflicting the skin, scalp and/or mucosae of a human subject, which comprise a therapeutically/cosmetically effective amount of at least one TNF-alpha antagonist. The compositions can further comprise an anti-inflammatory agent, such as diclofenac and salts thereof.

U.S. Pat. No. 5,804,572 to Blank et al. discloses compositions for regulating skin wrinkles and/or skin atrophy, wherein the compositions comprises salicylic acid, an anti-inflammatory agent and a pharmaceutically acceptable carrier. The anti-inflammatory agent can be diclofenac. The anti-inflammatory agent is preferably provided at a concentration of 0.5% to 5% of the composition.

U.S. Pat. No. 5,472,982 to Suzuki discloses an emulsified composition for treating the skin containing diclofenac sodium, a fatty acid and a dialkyl carboxylate, as essential components. The diclofenac sodium is preferably provided at a concentration of 0.5 to 5%.

A topical composition comprising 3 wt. % diclofenac, benzyl alcohol, PEG monomethyl ether, sodium hyaluronate, and water is available from Bioglan Pharmaceuticals as SOLARAZE The product is prescribed for the treatment of actinic keratosis. According to the FDA's Orange Book, SOLARAZE is covered by U.S. Pat. Nos. 5,639,738, 5,792,753, 5,852,002, 5,914,322, 5,929,048, and 5,985,850, all to Falk et al.

Despite the foregoing developments, it is desired to provide a method of treating photodamaged skin with a composition comprising diclofenac.

It is further desired to provide a method of treating rosacea with a composition comprising diclofenac.

It is still further desired to provide a method of treating acne with a composition comprising diclofenac.

All references cited herein are incorporated herein by reference in their entireties.

### BRIEF SUMMARY OF THE INVENTION

Accordingly, the invention provides a method of treating at least one dermatological disorder selected from the group consisting of photodamage-induced wrinkles, rosacea, and acne, said method comprising: (a) providing a composition comprising 1-3 wt. % diclofenac or a salt thereof, 0.5-1.5 wt. % benzyl alcohol, 10-30 wt. % PEG monomethyl ether, 0.1-3.0 wt. % sodium hyaluronate; and 70-80 wt. % water; and (b) applying the composition to skin of a person in need of said treating in an amount effective to treat the at least one dermatological disorder.

In certain embodiments, about 0.5-5 grams (preferably about 1-2 grams) of the composition is rubbed into the skin 1-2 times a day for an effective duration (e.g., at least 90 consecutive days).

In certain embodiments, the at least one dermatological disorder is photodamage-induced wrinkles.

In certain embodiments, photodamage-induced wrinkles are diminished.

In certain embodiments, the at least one dermatological disorder is rosacea, wherein excessive redness and/or inflammation of the skin are preferably reduced.

In certain embodiments, the at least one dermatological disorder is acne, wherein excessive redness and/or inflammation of the skin are preferably reduced.

In certain embodiments, the composition comprises about 3 wt. % diclofenac sodium, about 1.0 wt. % benzyl alcohol, about 20 wt. % PEG monomethyl ether, about 2.5 wt. % sodium hyaluronate and about 73.5 wt. % water.

Also provided is a method of reducing a severity of photodamage-induced wrinkles, said method comprising: (a) providing a composition comprising about 3 wt. % diclofenac sodium, about 1.0 wt. % benzyl alcohol, about 20 wt. % PEG monomethyl ether, about 2.5 wt. % sodium hyaluronate and about 73.5 wt. % water; and (b) repeatedly applying the composition to skin of a person in need of said treating in an amount effective to reduce the severity of the photodamage-induced wrinkles on the skin. In certain embodiments of this method, about 0.5-5 grams (preferably about 1-2 grams) of the composition is rubbed into the skin 1-2 times a day for an effective duration (e.g., at least 180 consecutive days).

Further provided is a method of treating rosacea, said method comprising: (a) providing a composition comprising about 3 wt. % diclofenac sodium, about 1.0 wt. % benzyl alcohol, about 20 wt. % PEG monomethyl ether, about 2.5 wt. % sodium hyaluronate and about 73.5 wt. % water; and (b) repeatedly applying the composition to skin of a person suffering from rosacea in an amount effective to treat the rosacea. In certain embodiments of this method, about 0.5-5 grams (preferably about 1-2 grams) of the composition is rubbed into the skin 1-2 times a day until redness and inflammation associated with the rosacea are reduced (i.e., for an effective duration, such as, e.g., at least 90 consecutive days).

Still further provided is a method of treating acne, said method comprising: (a) providing a composition comprising about 3 wt. % diclofenac sodium, about 1.0 wt. % benzyl alcohol, about 20 wt. % PEG monomethyl ether, about 2.5 wt. % sodium hyaluronate and about 73.5 wt. % water; and (b) repeatedly applying the composition to skin of a person suffering from rosacea in an amount effective to treat the acne. In certain embodiments of this method, about 0.5-5 grams (preferably about 1-2 grams) of the composition is rubbed into the skin 1-2 times a day until redness and inflammation associated with the acne are reduced (i.e., for an effective duration, such as, e.g., at least 90 consecutive days).

### DETAILED DESCRIPTION OF THE INVENTION

Applicants have now developed compositions, (combinations and formulations) which are topically applied to the skin and/or exposed tissue of a human and which are quickly transported in dosage amounts percutaneously (intracutaneously) at a site in need of treatment, (site of pathology and/or trauma) best targeting the epidermis and subsequently remaining (accumulating) at the site for a prolonged period of time. The compositions subsequently clear through the lymphatics thereby bringing dosage amounts of the compositions to the lymphatics for the treatment of disease and conditions in the lymphatics.

These compositions, (combinations and formulations) employ, combine, or incorporate (as the case may be) a plurality of effective non-toxic dosage amounts, each dosage amount comprising an effective non-toxic dosage amount of a drug for example a drug which inhibits prostaglandin synthesis for example an NSAID and an effective non-toxic dosage amount of a form of hyaluronic acid (preferably hyaluronic acid or salt thereof) for the transport of the drug to the site of the pathology and/or trauma. Suitable dosage amounts of the composition may be removed from a container (for example a tube or jar) and administered (for example, applied). Thus according to one aspect of the invention these pharmaceutical compositions (combinations and formulations) comprise a plurality of effective non-toxic dosage amounts for administration to the skin and/or exposed tissue of a human in need of treatment, each such dosage amount comprising a therapeutically effective non-toxic (to the patient) dosage amount of a drug to treat a disease and/or condition for example a drug which inhibits prostaglandin synthesis, preferably being a non-steroidal anti-inflammatory drug (NSAID), for example, diclofenac, indomethacin, naproxen, and ± tromethamine salt of ketorolac (sold under the trademark TORADOL) and an effective non-toxic dosage amount (for example in excess of 5 mg per cm² (square centimeter) of skin or exposed tissue to which the dosage amount of the composition is to be applied) of hyaluronic acid and/or salts thereof (for example the sodium salt) and/or homologues, analogues, derivatives, complexes, esters, fragments, and/or sub units of hyaluronic acid (preferably hyaluronic acid and/or salts thereof) to transport (to facilitate or cause the transport of) the drug to the site of the pathology and/or trauma of the disease or condition. These compositions may be applied topically to treat diseases and conditions of the skin and/or exposed tissue at the site of the trauma and/or pathology, (for example, basal cell carcinoma, the precancerous, often recurrent, actinic keratoses lesions, fungal lesions, “liver” spots and like lesions (found for the most part in the epidermis), squamous cell tumors, metastatic cancer of the breast to the skin, primary and metastatic melanoma in the skin, malignancies and/or tumors in the skin, genital warts (condyloma acuminata), cervical cancer, and HPV (Human Papilloma Virus) including HPV of the cervix, psoriasis (both plaque-type psoriasis and nail bed psoriasis), corns on the feet and hair loss on the head of pregnant women). The results of the treatment with suitable, dosage amounts taken from these compositions (combinations and formulations) have been in some instances quite dramatic--difficult situations have been successfully treated and resolved.

Furthermore, application of the dosage amounts of the compositions, combinations and formulations are, systemic independent (there is a lack of a blood level of the drug for example NSAID), are quick to penetrate into the skin to the site of the trauma and/or pathology because the effective dosage amount of the form of hyaluronic acid transports (facilitates or causes the transport of) the drug (for example NSAID) particularly to the epidermis where the composition, combination or formulation accumulates and remains for prolonged periods. The compositions subsequently clear through the lymphatics and are available for the treatment of disease and conditions of the lymphatics.

In this regard effective amounts of the form of hyaluronic acid exceeds in the order of about 5 mg per square cm (cm²) of the area of for example the skin and/or exposed tissue to which the dosage amounts of the composition is to be applied.

Thus, according to another aspect of the invention, Applicants have provided topically applicable percutaneous (intracutaneous) penetrating (best targeting the epidermis) systemic independent acting (not acting essentially through the blood) pharmaceutical compositions (combinations and formulations) comprising a plurality of dosage amounts each comprising, together with pharmaceutical excipients suitable for topical application, a therapeutically effective (to treat and to assist to resolve diseases and conditions of the skin and exposed tissue (for example basal cell carcinoma, the precancerous, often recurrent, actinic keratoses lesions, fungal lesions, “liver” spots and like lesions (found for the most part in the epidermis), squamous cell tumors, metastatic cancer of the breast to the skin, malignancies and/or tumors in the skin primary and metastatic melanoma in the skin, genital warts (condyloma acuminata), cervical cancer, and HPV (Human Papilloma Virus) including HPV of the cervix, psoriasis (both plaque-type psoriasis and nail bed psoriasis), corns on the feet and hair loss on the head of pregnant women), non-toxic (to the patient) dosage amount of a drug for example which inhibits prostaglandin synthesis, preferably a non-steroidal anti-inflammatory drug (NSAID), for example, diclofenac, indomethacin, naproxen, and (±) tromethamine salt of ketorolac (sold under the trademark TORADOL) and an effective non-toxic amount of hyaluronic acid and/or salts thereof (for example, the sodium salt) and/or homologues, analogues, derivatives, complexes, esters, fragments, and/or sub-units of hyaluronic acid (preferably hyaluronic acid and salts thereof) to transport (facilitate or cause the transport of) the drug (for example NSAID's) rapidly to the site in the skin (for example epidermis) and/or exposed tissue of the disease or condition into the tissue to remain there for a prolonged period of time to assist to treat and assist to resolve the disease or condition for example by blocking prostaglandin synthesis.

Preferably, the pharmaceutical compositions are therapeutically effective to treat and/or prevent at least one of photodamage, acne and rosacea in a mammal (e.g., a human).

Effective dosage amounts of the form of hyaluronic acid to facilitate or cause the transport of the drug into the skin and/or exposed tissue by the form of hyaluronic acid exceeds about 5 mg-10 mg in the dosage amount administered (applied and rubbed in) for each 1 cm² of skin and/or exposed tissue area of the disease or condition (for example, photodamage, acne and/or rosacea) to which the dosage amount is applied. The dosage amount applicable will depend upon the surface area of the skin and/or exposed tissue in which the condition or disease exists. Thus if the disease or condition occupies about 0.5 cm², in excess of about 2.5 mg of the form of hyaluronic acid would be used (applied and rubbed in). In the same way if the area is 2 cm², the amount of the form of hyaluronic acid preferably exceeds about 10-20 mg of the dosage amount of the formulation or composition applied. Preferred forms of the hyaluronic acid (for example hyaluronic acid and the sodium salt thereof) have molecular weights less than about 750,000 daltons (for example about 150,000 to about 225,000 daltons) to transport the medicine in the skin and/or exposed tissue. While higher molecular weights of the hyaluronic acid and forms thereof may be used to penetrate the skin and/or exposed tissue and transport the medicines or drugs, where the molecular weight of the hyaluronic acid chosen for use is very large, it is preferred that the form of hyaluronic acid is autoclaved, to break down the hyaluronic acid to fragments of lesser molecular weight or if feasible diluted to permit administration and ensure no coagulation on or in the skin. Where the molecular weight of the form of hyaluronic acid being employed is large, the concentration of the form of the hyaluronic acid in the composition may for example be reduced (for example to less than about 3%) dependent on the molecular weight.

The blockage of prostaglandin synthesis by the transported drug (for example NSAIDS) then unblocks the macrophages and permits the macrophages of the patient proximate the lesion (for example, the basal cell carcinoma) to destroy the lesion or condition. Treatment by dosage amounts of the composition (formulation and/or combination) eliminates the condition without recurrence, even where the lesion has recurred a number of times after unsuccessful treatments according to the prior art.

Although diclofenac is the preferred NSAID, other non-steroidal anti-inflammatory drugs (NSAIDS) may be used such as other propionic acid derivatives, Ibuprofen, acetylsalicylic acid, piroxicam and flunixin.

When dosage amounts of such compositions, combinations and formulations are applied to the site of the disease or condition over a period of time (for example, for a period of 2-4 weeks 3 times daily) the abnormal skin condition is completely resolved and disappears, or less preferably, is at least prevented (or in the case of photodamage, delayed) from increasing in severity.

Thus according to another aspect of the invention there is provided a pharmaceutical composition from which dosage amounts may be taken for application to the skin and/or exposed tissue, the pharmaceutical composition comprising in a form for application to a human a plurality of dosage amounts of medicine and/or therapeutic agent to treat a disease or condition in a human and a plurality of dosage amounts of hyaluronic acid and/or salts and/or homologues, analogues, derivatives, complexes, esters, fragments, and/or sub-units of hyaluronic acid such that when dosage amounts of the pharmaceutical composition are taken from the composition, the amount of the medicine and/or therapeutic agent comprises an effective non-toxic dosage amount of the medicine to treat the disease or condition in the skin and/or exposed tissue in a human and the amount of the form of hyaluronic acid in the dosage amount is present in an effective amount to transport (facilitate or cause the transport of) the medicine and/or therapeutic agent intradermally (percutaneously, intercutaneously, intracutaneously) into the skin (preferably to the epidermis and dermis) and/or exposed tissue of a human to the site of a pathology and/or trauma. The effective amount of the form of hyaluronic acid has a molecular weight and concentration to transport the medicine (drug) and/or therapeutic agent to the site of trauma and/or pathology in the skin and/or exposed tissue. In this regard the preferred amount of the form of hyaluronic acid in each dosage amount exceeds 5 mg/cm² and preferably the molecular weight is less than about 750,000 daltons, (in one embodiment about 150,000 to about 225,000 daltons) in some embodiments with a concentration of between about 1 and 3%, preferably concentrations of between about 2 to about 3% by weight. Where forms of hyaluronic acid are used having greater molecular weights, they are preferably cleaved and/or diluted to smaller concentrations, to facilitate or cause the transport of the medicine and/or therapeutic agent.

According to another aspect of the invention there is provided a pharmaceutical composition (for example a gel or cream) from which dosage amounts may be taken and applied to the skin to treat a disease or condition in humans, for example as discussed above, the pharmaceutical composition comprising:

(1) a medicinal and/or therapeutic agent suitable for treating a disease or condition in the skin and/or exposed tissue in humans, for example a drug which inhibits prostaglandin synthesis (for example an NSAID); and

(2) hyaluronic acid and/or salts thereof and/or homologues, analogues, derivatives, complexes, esters, fragments, and sub-units of hyaluronic acid, in a form suitable for administration to the skin and/or exposed tissue in humans; characterized in that an effective non-toxic dosage amount comprising components (1) and (2) taken and administered from said composition (i) is available in the skin and/or exposed tissue upon administration to treat said disease or condition in humans by penetration at the site to be treated to the site of trauma and/or pathology, and (ii) comprises an effective non-toxic dosage amount of component (2) effective to transport (facilitate or cause the transport of) component (1) immediately upon administration percutaneously into the skin (preferably the epidermis) to the site to be treated for example the site of trauma and/or pathology where it remains for a prolonged time, accumulating there and from which it is discharged via the lymphatic system.

Therefore according to another aspect of the invention a pharmaceutical composition is provided comprising:

(1) a medicinal and/or therapeutic agent which for example inhibits prostaglandin synthesis in a therapeutically effective amount to treat a disease or condition of the skin and/or exposed tissue;

and (2) hyaluronic acid and/or salts thereof and/or homologues, analogues, derivatives, complexes, esters, fragments, and subunits of hyaluronic acid, characterized in that said composition

(a) is in a dosage form (for example a gel or cream) which is suitable for administration to the skin and/or exposed tissue;

and (b) is in such an amount and in such form that (i) component (1) is in an effective dosage amount to treat said disease or condition by penetration at the site of the skin and/or exposed tissue to be treated for example the basal cell carcinoma and other lesions; and (ii) component (2) is immediately available to transport (facilitate or cause the transport of) component (1) to the site of trauma and/or pathology to be treated, percutaneously into the skin (or exposed tissue) where the composition resides and accumulates for a prolonged period, and which component (2) is in an effective non-toxic dosage amount to transport (facilitate or cause the transport of) component (1) upon administration, percutaneously into the skin or exposed tissue to the site of the trauma and/or pathology. Preferably the form of hyaluronic acid in the composition comprises hyaluronic acid and/or salts thereof. An effective amount of the form of hyaluronic acid exceeds about 5-10 mg per square centimeter (cm²) of skin and/or exposed tissue to which it is to be applied.

According to another aspect of the invention there is provided the use of

(1) a medicinal and/or therapeutic agent for example which inhibits prostaglandin synthesis, and (2) hyaluronic acid and/or salts, thereof and/or homologues, analogues, derivatives, complexes, esters, fragments, and subunits of hyaluronic acid, in the manufacture of a pharmaceutical composition for treating a disease or a condition (for example those discussed above) of the skin and/or exposed tissue in a therapy wherein dosage amounts taken from the composition each comprise:

(1) a therapeutically effective amount of said medicinal and/or therapeutic agent and

(2) a therapeutically effective amount of the hyaluronic acid and/or salts thereof and/or homologues, analogues, derivatives, complexes, esters, fragments, and sub-units of hyaluronic acid, the pharmaceutical composition being characterized in that for each dosage amount taken from the pharmaceutical composition, the amount of component (2) is immediately available to transport component (1) percutaneously to the site of trauma and/or pathology for example into the epidermis where the composition accumulates and remains for a prolonged period, at the site of the skin or exposed tissue to be treated, and component (2) is in an effective non-toxic amount to transport (facilitate or cause the transport of) component (1) into the skin or exposed tissue (for example into the epidermis). Preferably component (2) is hyaluronic acid and/or salts thereof and preferably the dosage amount of component (2) in the amount of the composition taken from the composition (to be taken from the composition) and applied to the skin or exposed tissue is a dose amount greater than about 5-10 mg per cm² of skin, and/or exposed tissue to which the dosage amount is to be applied.

The pharmaceutical composition will normally include pharmaceutically compatible excipients to provide a form for ease of administration to the skin and/or exposed tissue for transport into the epidermis. For example a suitable dosage amount of a gel may be squeezed from a tube as a ribbon of gel “X” cm long (which dosage amount (in the form of the ribbon “X” cm long)) contains the effective non-toxic dosage amounts of the drug and form of hyaluronic acid. Or a dosage amount of cream packaged in a jar may be scooped from the jar by a measuring device or by “two fingers” in a suitable amount (for example in a spoon containing a premeasured volume or an amount about half the “length of the fingers”). Each of the dosage amounts selected comprises the effective amounts of drug (for example NSAID) and effective amount of the form of hyaluronic acid (for example hyaluronic acid and/or salts thereof). In this way the patient may “squeeze” or “scoop” or “what have you” the appropriate dosage amount and apply (rub in) the dosage amount onto the skin and/or exposed tissue for transport into the epidermis.

Thus, according to another aspect of the invention, a method of treating a disease and/or condition of the skin or exposed tissue, for example photodamage, acne and/or rosacea, in a human is provided comprising administering topically to human skin and/or exposed tissue an effective non-toxic dosage amount of a composition comprising, together with pharmaceutical excipients suitable for topical application to the skin and/or exposed tissue, for example in the form of a gel or cream (to give the composition definition and form so that specific dosage amounts are easily selected or taken for administration (for example squeezed from a tube or scooped from a jar and rubbed into the skin or exposed tissue), a therapeutically effective (to treat and to assist to resolve the disease or condition for example basal cell carcinoma or other lesion), non-toxic (to the patient) dosage amount of a drug for example which inhibits prostaglandin synthesis, for example a non-steroidal anti-inflammatory drug (NSAID), for example, diclofenac, indomethacin, naproxen, and (±) tromethamine salt of ketorolac (sold under the trademark TORADOL) and an effective non-toxic dosage amount of hyaluronic acid and/or salts thereof (for example, the sodium salt) and/or homologues, analogues, derivatives, complexes, esters, fragments, and/or sub-units of hyaluronic acid (preferably hyaluronic acid and salts thereof) to transport (facilitate or cause the transport of) the drug (for example NSAID) into the skin or exposed tissue to the site of the disease or condition to be treated percutaneously, (to the site of trauma and/or pathology), for example into the epidermis, where the form of hyaluronic acid and medicine accumulates and remains for a prolonged period of time thereby for example blocking prostaglandin synthesis in the skin or exposed tissue. The form of hyaluronic acid is then cleared through the lymphatics (lymphatics system).

Thus, according to another aspect of the invention, the treatment may employ the use of the composition, formulation or combination for the treatment of the diseases and conditions aforesaid as for example by applying dosage amounts of the composition, formulation or combination a number of times daily (for. example, 3 times daily) for a period of time, for example, 2-4 weeks to clear the disease, lesion or condition. Each dosage amount applied will depend upon the size of the lesion or condition on the skin or exposed tissue. For example, a suitable dosage amount may include 5-10 mg of the form of hyaluronic acid per 1 cm² skin area or exposed tissue area. One such formulation may comprise 3% (by weight) diclofenac in a 2.5% (by weight) hyaluronic acid (sodium hyaluronate--molecular weight 661,600) gel formulation, with the excipients being glycerine (5%), benzyl alcohol (3%) (acting in part as a solubilizer and preservative), and sterile water (the balance) in a 50 gm tube of the composition (a plurality of dosage amounts) whose tube O.D. (outer diameter) of the opening through which the gel formulation is discharged from the tube is 8 mm and whose I.D. (inner diameter) of the opening is 4 mm. Therefore a ribbon 2-3 cm in length, squeezed from a tube gives about 5 mg-7.5 mg of hyaluronic acid for application to a skin or exposed tissue surface area of 1-1.5 cm² with an effective dosage amount of diclofenac. While greater amounts squeezed from the tube may be applied, the application of substantial excessive dosage amounts to the skin and/or exposed tissue may saturate the skin or exposed tissue and thus the epidermis. (There is therefore no more room for the composition to pass between the cells and therefore further applications at that time will not provide additional benefit). Where pain relief is also required additional dosage amounts, for example in excess of about 10 mg of the hyaluronic acid taken from the same pharmaceutical composition applied per/cm² of surface area of the skin or exposed tissue may be required to be applied.

Another formulation may comprise 3% (by weight) diclofenac in a 2.5% (by weight) hyaluronic acid (sodium hyaluronate--molecular weight 679,000) gel formulation (also in a tube) with excipients being benzyl alcohol (1%) (a preservative), methoxypolyethylene glycol 350 (20% by weight) (a solubilizer), and sterile water (the balance).

While the above compositions, combinations and formulations are proposed, provided there is sufficient amounts of the form of the hyaluronic acid (for example, sodium hyaluronate) in the dosage amounts applied to the skin and/or exposed tissue to facilitate or cause the percutaneous (intracutaneous) transport of the drug for example which inhibits prostaglandin synthesis, preferably an NSAID (for example, diclofenac) to block prostaglandin synthesis, then the formulations may be of any suitable form, for example, a 1% lotion of hyaluronic acid with NSAID, or a cream or gel or any other suitable form.

Therefore according to another aspect of the invention, there are provided containers (for example tubes and jars) containing compositions comprising a plurality of dosage amounts of the drug and form of hyaluronic acid, each dosage amount comprising an effective non-toxic dosage amount of the drug and an effective non-toxic dosage amount of the form of hyaluronic acid (preferably sodium hyaluronate having molecular weight less than about 750,000 daltons) to transport the drug into the skin and/or exposed tissue. In some embodiments, means are provided to assist the removal from the container of an effective dosage amount of the composition in the container for use to apply to the skin or exposed tissue at the site of trauma and/or pathology to treat the disease and/or condition (for example mouth opening of a tube to control the amount discharged from the tube).

Furthermore, because there is little concern with respect to the toxicity or adverse effects of the use of, for example, the NSAIDs with the hyaluronic acid in the compositions of this invention the NSAID may be combined as needed (after solubilizing (if required) of the NSAID in a suitable solubilizer) with the form of the hyaluronic acid.

Therefore according to another aspect of the invention, percutaneous (intercutanous) delivery of a therapeutically effective dosage amount of a drug (in a composition, combination or formulation) and which drug for example inhibits prostaglandin synthesis, preferably being a non-steroidal drug (NSAID) is provided. In this regard the drug is transported to the site of, on, or in the skin and/or exposed tissue of trauma and/or pathology to treat the disease or condition for example photodamage, rosacea and/or acne in a mammal (human). The delivery may comprise topically administering (to the skin or exposed tissue site of for example the basal cell carcinoma or other lesion) a therapeutically effective non-toxic (to the patient) dosage amount of a composition comprising a drug for example which inhibits prostaglandin synthesis, preferably an NSAID (non-steroidal anti-inflammatory drug), for example, diclofenac, indomethacin, naproxen, and (±) tromethamine salt of ketorolac (sold under the trademark Toradol™), and an effective non-toxic amount of hyaluronic acid and/or salts thereof and/or homologues, analogues, derivatives, complexes, esters, fragments, and sub-units of hyaluronic acid, preferably hyaluronic acid and salts thereof, sufficient to transport, (facilitate or cause the transport of), the drug for example NSAID percutaneously (to for example the epidermis) to the site of the trauma and/or pathology in for example the epidermis, for example the basal cell carcinoma (or other lesion), to be treated for example to block the synthesis of prostaglandins.

Delivery may be also accomplished by the same amount of the form of hyaluronic acid, of other drugs percutaneously (intercutaneously) to the skin and exposed tissue by application and rubbing in of an effective non-toxic dosage amount of the formulation or composition comprising an effective non-toxic dosage amount of the drug and an effective non-toxic dosage amount of the form of hyaluronic acid for the transport of the drug percutaneously into the skin or exposed tissue to the epidermis where the dosage amount of the composition is accumulated and remains for a prolonged period of time before the form of hyaluronic acid is cleared through the lymphatics.

Thus, according to another aspect of the invention, use of a composition, combination or formulation is provided to treat a disease or condition for example basal cell carcinoma (or other lesion), by the application of the composition, combination or formulation, the amount of the composition, combination and formulation administered comprising together with pharmaceutical excipients suitable for topical application, a therapeutically effective (to treat and assist to resolve a disease or condition for example, basal cell carcinoma), non-toxic (to the patient) amount of a drug for example which inhibits prostaglandin synthesis preferably a non-steroidal anti-inflammatory drug (NSAID), for example, diclofenac, indomethacin, naproxen, and (±) tromethamine salt of ketorolac (sold under the trademark TORADOL) administered together with, or carried in, an effective dosage amount of hyaluronic acid and/or salts thereof (for example, the sodium salt) and/or homologues, analogues, derivatives, complexes, esters, fragments, and/or sub-units of hyaluronic acid (preferably hyaluronic acid and salts thereof) effective to transport the drug for example the NSAID (to facilitate or cause the transport of the drug for example NSAID) percutaneously into the skin especially the epidermis at the site of the disease or condition for example photodamage, rosacea and/or acne to be treated, thereby, if an NSAID, blocking prostaglandin synthesis to enable the macrophages (and N.K. cells) to resolve the disease or condition for example basal cell carcinoma or other lesion.

Applicants postulate that the hyaluronic acid and/or salts thereof and/or the homologues, analogues, derivatives, complexes, esters, fragments, and/or sub units of hyaluronic acid facilitate or cause the transport of the drug for example which blocks prostaglandin synthesis (preferably an NSAID) to the site of prostaglandin synthesis to block prostaglandin synthesis. Applicants' compositions and dosage amounts of their compositions and the use of their compositions and dosage amounts of their compositions, at the same time, abate pain that the patient is experiencing at the paccinian nerve bundles (superficial nerve bundles) at the site of the trauma and/or pathology on/in the exposed tissue and/or skin.

Thus, according to another aspect of the invention, a method of abating pain in the skin and/or exposed tissue for example suffering a disease or condition (for example those discussed above), and a composition from which dosage amounts may be taken and applied (rubbed in) which is useful for abating such pain are provided, the method comprising administering (rubbing on) an effective dosage amount of the composition to the skin and/or exposed tissue, and the composition comprises a plurality of dosage amounts, each comprising an effective non-toxic dosage amount of an NSAID and an effective non-toxic dosage amount of the hyaluronic acid and/or salts thereof and/or homologues, analogues, derivatives, complexes, esters, fragments, and/or subunits of hyaluronic acid (preferably hyaluronic acid and salts thereof), for example amounts exceeding 10-20 mg per square cm (cm²) of skin or exposed tissue to which it is applied, for percutaneous transport of the NSAID by the form of hyaluronic acid into the epidermis proximate the paccinian nerve bundles (superficial nerve bundles at the end of the nerves) to abate pain. Thus, according to another aspect of the invention, compositions are provided for use to relieve pain from which dosage amounts of the composition comprising dosage amounts of the NSAID and form of hyaluronic acid are taken.

By way of example and to illustrate the facilitation of the delivery or transport of a chemical to a site in a human, when ethyl alcohol is injected directly into a tumor and sonographic (ultrasound) assessment is made, it is not dispersed throughout the tumor. When the ethyl alcohol to be administered into a tumor is carried by hyaluronic acid and/or salts thereof, sonographic assessment of the tumor demonstrates the dispersion of the ethyl alcohol throughout the tumor.

While Applicants postulate that the hyaluronic acid facilitate or causes the transport and delivery, Applicants' invention may be used as described irrespective of the actual method of operation of the hyaluronic acid and/or salts thereof and/or the homologues, analogues, derivatives, complexes, esters, fragments and sub-units of hyaluronic acid.

The combination of hyaluronic acid and salts thereof and other forms with drugs for example that inhibit prostaglandin synthesis, for example NSAIDs, alters their distribution and performance in the skin and/or exposed tissue particularly the epidermis (the combinations and formulations being systemic independent), and produces an unusual targeting for underperfused skin and/or pathological tissue in the skin (site of trauma and/or pathology). The application may be made as required with the amount depending upon the condition of the skin or exposed tissue.

As a major amount of soluble indomethacin may be incorporated into the formulation, or composition, the indomethacin may be solubilized using n-methyl glucamine at a dilution of 5 mg/ml of n-methyl glucamine (NMG). This substance is then passed through a 22 micron Milipore filter to produce sterility. This material is non-toxic at 16 fold the therapeutic dose in animals (with hyaluronic acid) and for this reason was considered appropriate to be used in human conditions. Thus, INDOCID solubilized in NMG may be administered with hyaluronic acid topically for percutaneous penetration at, for example, varying doses. The solution of indomethacin and NMG may be mixed with, for example, LIFECORE hyaluronic acid in dosage amounts discussed above. This produces an appropriate mixture and can be administered safely.

When the NSAID, for example indomethacin (dissolved in n-methyl glucamine) or other NSAID, is applied topically in an effective dosage amount from a composition or formulation also including the effective dosage amount of the form of hyaluronic acid, no major toxic side effects occur, such as gastro-intestinal distress, neurological abnormalities, depression, etc., even at elevated amounts of indomethacin (if necessary). (This may be in part because of the clearing of the hyaluronic acid through the lymphatic system from the site). In addition, the responses that have been observed are dramatic when the drug for example NSAID (for example diclofenac) is combined with hyaluronic acid, demonstrating clearly that the combination is now “targeting” to the site of pathology or trauma, or pathological tissue. Furthermore, patients using the formulations and combinations of drug (for example NSAID)—hyaluronic acid (sodium hyaluronate) (for example, diclofenac or indomethacin and hyaluronic acid), experience dramatic relief of pain immediately.

Thus, Applicants believe that the use of the. NSAID, for example with hyaluronic acid (sodium hyaluronate), deblocks the macrophages (and N.K. cells (Natural Killer Cells) thought to be immature macrophages) by preventing enzymatic production of prostaglandin, which blocks macrophage (and N.K. cell) functioning. The hyaluronic acid (and salt and other forms) not only enhances the activity of the drug (NSAID) but also reduces any side effects and toxicity that is associated with the use of the prostaglandin synthesis inhibitors. When effective dosage amounts of compositions, formulations and combinations containing effective dosage amounts of the drugs for example, (NSAIDs (for example, diclofenac)) and effective dosage amounts of, for example, hyaluronic acid or the sodium salt thereof, are applied to the skin to be treated, the condition of the skin improves.

Applicants also postulate that when the combination or formulation is applied to the disease or condition (for example, photodamage, rosacea and/or acne), the hyaluronic acid passes between the cells (in the stratum corneum and epidermis to the dermis depending on amounts) to the areas of trauma and/or pathology deficient in hyaluronic acid (or forms thereof), transporting, taking, drawing, carrying or pulling the NSAID with it to the sites of prostaglandin synthesis, penetrating to inhibit prostaglandin synthesis until the space between the cells is saturated. The NSAID now being proximate the Paccinian nerve bundle (superficial nerve bundles at the end of the nerves) gives pain relief. The macrophages (which had been previously blocked) are unblocked and act to destroy the disease or condition for example basal cell carcinoma, actinic keratoses lesion, or other disease or lesion. Furthermore, the effective non-toxic dosage amount of the composition, combination or formulation, comprising the effective dosage amount of the form of hyaluronic acid and the effective dosage amount of NSAID passing through the stratum corneum to the epidermis and to the dermis (if a sufficient amount of the form of hyaluronic acid is present), passes into the skin, accumulating and staying longer in the skin at the site of the trauma and/or pathology. Therefore, after having had an immediate effect at the site of trauma and/or pathology (for example, relieving pain and acting on the basal cell carcinoma, actinic keratoses and other disease, condition or lesion), the NSAID-hyaluronic acid combination continues to accumulate at the site in need of treatment and thereafter clears through the lymphatic system.

Thus according to another aspect of Applicant's invention, Applicants' compositions, formulations and combinations quickly penetrate on application through the stratum corneum into the epidermis (to the dermis) by the form of hyaluronic acid transporting the NSAID, to the site of trauma and/or pathology where the amounts applied accumulate and remain for a prolonged time for treatment.

Fifteen minutes after application of one of Applicants' preferred formulations, about three times the amount of Applicants' formulation has penetrated into the skin (particularly the epidermis) than formulations and combinations not containing hyaluronic acid or effective dosage amounts of hyaluronic acid, but containing the same drug. Furthermore, the drug and hyaluronic acid accumulate and remain at the site in need of treatment for a longer period of time.

Thus according to another aspect of the invention, non-toxic effective dosage amounts of forms of hyaluronic acid (preferably sodium hyaluronate) and effective non-toxic dosage amounts of a drug may be administered in compositions to sites of trauma or pathology, on/in the skin and/or exposed tissue (for example the epidermis) by the application of the effective non-toxic dosage amount of the composition comprising an effective non-toxic dosage amount of a drug (for example an NSAID) and an effective non-toxic dosage amount of a form of hyaluronic acid (for example sodium hyaluronate) to the skin or exposed tissue whereby the forms hyaluronic acid transport the drug percutaneously to the site of trauma and/or pathology where the composition accumulates and remains for a prolonged period of time thereby retaining the drug at the site of trauma and/or pathology (for example the epidermis) for the treatment of the condition or disease and the reduction of pain.

Thus according to another aspect of the invention, Applicants have provided compositions (formulations and combinations) (including pharmaceutical excipients suitable for topical application) from which effective non-toxic (to the patient) dosage amounts of a drug (for example an NSAID) to treat and to assist to resolve diseases and conditions of the skin and/or exposed tissue (for example photodamage, rosacea and/or acne), and effective non-toxic dosage amounts of hyaluronic acid and/or salts thereof (for example, the sodium salt) and/or homologues, analogues, derivatives, complexes, esters, fragments, and/or sub-units of hyaluronic acid (preferably hyaluronic acid and salts thereof) sufficient to transport (to facilitate or cause the transport of) the drug, for example NSAID, are taken for application, to a site in the skin (for example epidermis) or exposed tissue having a disease or condition for percutaneous transport into the skin and/or exposed tissue to accumulate and remain there for a prolonged period of time to for example block prostaglandin synthesis. Thus an effective dosage amount of the composition or formulation or combination penetrates quickly into the skin, for example by the hyaluronic acid transporting the NSAID or causing the NSAID to be transported for example to the epidermis of the skin, accumulates there and remains there for a prolonged period of time, thereby accumulating the drug and forms of hyaluronic acid in the skin (particularly ₜhe epidermis).

Thus according to another aspect of the invention, a method of accumulating a drug and a form of hyaluronic acid in skin and/or exposed tissue is provided comprising topically administering a therapeutically effective non-toxic dosage amount of a composition comprising pharmaceutical excipients suitable for topical applications, an effective non-toxic (to the patient) dosage amount of a drug for example which inhibits prostaglandin synthesis, preferably a non-steroidal anti-inflammatory drug (NSAID), for example, diclofenac, indomethacin, naproxen, and (±) tromethamine salt of ketorolac (sold under the trademark TORADOL) (to treat and to assist to resolve the disease and conditions of the skin and exposed tissue (for example photodamage, rosacea and/or acne), and an effective non-toxic dosage amount of hyaluronic acid and/or salts thereof (for example, the sodium salt) and/or homologues, analogues, derivatives, complexes, esters, fragments, and/or sub-units of hyaluronic acid (preferably hyaluronic acid and salts thereof) effective to transport (to facilitate or cause the transport of) the drug (for example NSAID) percutaneously to the site in the skin (for example epidermis) or exposed tissue of the disease or condition to accumulate and remain there for a prolonged period of time for example to block prostaglandin synthesis.

According to another aspect of the invention, a method of quickly delivering a drug to the skin or exposed tissue, particularly the epidermis, and maintaining the drug therein for a prolonged period of time is provided, the method comprising topically administering (for example rubbing in) an effective non-toxic dosage amount of a composition comprising pharmaceutical excipients suitable for topical application, a therapeutically effective (to treat and assist to resolve the disease and/or condition of the skin and exposed tissue (for example photodamage, rosacea and/or acne), non-toxic (to the patient) dosage amount of a drug for example which inhibits prostaglandin synthesis, preferably a non-steroidal anti-inflammatory drug (NSAID), for example, diclofenac, indomethacin, naproxen, and (±) tromethamine salt of ketorolac (sold under the trademark TORADOL) and an effective non-toxic dosage amount of hyaluronic acid and/or salts thereof (for example, the sodium salt) and/or homologues, analogues, derivatives, complexes, esters, fragments, and/or sub-units of hyaluronic acid (preferably hyaluronic acid and salts thereof) sufficient to transport (to facilitate or cause the transport of) the drug for example the NSAID percutaneously to the site of the trauma and/or pathology in the skin (for example epidermis) or exposed tissue, for remaining there for a prolonged period of time (for example in the epidermis and dermis) to for example block prostaglandin synthesis. Suitable amounts of the form of hyaluronic acid may comprise in excess of 5 mg per cm² in a form which transports the drug (for example molecular weights of the form of hyaluronic acid being less than about 750,000 Daltons or if at substantially greater molecular weights, diluted (to reduce) the concentration or autoclaved or cleaved if required to reduce the size of the molecules.

According to another aspect of the invention, a method of controlling the unloading of a drug from the skin or exposed tissue into the lymphatic system comprises delivering (transporting) an amount of drug into the skin or exposed tissue by an effective non-toxic dosage amount of a form of hyaluronic acid and/or salts thereof and/or homologues, analogues, derivatives, complexes, esters, fragments, and/or sub-units of hyaluronic acid to the skin (epidermis) or exposed tissue to control the unloading of the drug into the lymphatic system (for example by the application of greater than 5 mg/cm²) of the form of hyaluronic acid.

Thus according to another aspect of the invention a composition is provided which when administered to a human by preferably administration to the skin and/or exposed tissue of a human, unloads its contents into the lymphatic system, the composition comprising an effective non-toxic dosage amount of a drug (for example an NSAID or an anti-cancer drug (Novantrone) and an effective non-toxic amount of hyaluronic acid and/or salts thereof and/or homologues, analogues, derivatives, complexes, esters, fragments and/or sub-units of hyaluronic acid (for example at least about 5-10 mg/cm² of skin or exposed tissue). Thus the composition is made up of a plurality of such dosage forms (for example a cream or lotion or gel).

Thus according to another aspect of the invention, a new composition for treating diseases via the lymphatic system is provided comprising a plurality of effective non-toxic dosage amounts of the composition, each dosage amount comprising hyaluronic acid and/or salts thereof and/or homologues, analogues, derivatives, complexes, esters, fragments and/or sub-units of hyaluronic acid for passing into the lymphatic system and a therapeutic effective amount of medicine for treatment of a disease (which disease may be in the lymphatic system).

According to another aspect of the invention, the composition may be for application to the skin or exposed tissue.

According to another aspect of the invention, a composition is provided from which effective dosage amounts may be taken and administered, each effective dosage amount of the composition comprising an effective non-toxic dosage amount of hyaluronic acid and/or salts thereof and/or homologues, analogues, derivatives, complexes, esters, fragments and/or sub-units for transporting a therapeutically effective non-toxic dosage amount of a medicine and/or therapeutic agent (for example an NSAID) in the composition into the skin and/or exposed tissue when applied thereto to an area of pathology and/or trauma then into the lymphatic system, the dosage amount being essentially systemic independent such that substantial amounts do not enter the blood system prior to clearing (passing) into the lymphatic system. Preferably the amount of the form of hyaluronic acid in each dosage amount administered is greater than about 5-10 mg/cm² and the molecular weight is less than about 750,000 daltons.

The invention will be illustrated in more detail with reference to the following Examples, but it should be understood that the present invention is not deemed to be limited thereto.

### EXAMPLES

### Example 1

A human having photodamaged skin is treated with the prescription medication SOLARAZE, obtained from Bioglan Pharmaceuticals, Malvern, Pa., USA. SOLARAZE comprises about 3 wt. % diclofenac sodium, about 1.0 wt. % benzyl alcohol, about 20 wt. % PEG monomethyl ether, about 2.5 wt. % sodium hyaluronate and about 73.5 wt. % water. Approximately 1-2 grams is rubbed into the skin to be treated 1-2 times a day for a mimimum period of 180 days. After this course of treatment, the treated skin will tighten and fine wrinkles will be diminished (e.g., in number, length, width, depth, etc.). In addition, lentigines or lentigos (i.e., liver spots) will also be diminished (e.g., faded).

### Example 2

A human suffering from rosacea is treated with SOLARAZE. Approximately 1-2 grams is rubbed into the skin to be treated 1-2 times a day for a period of 90 days. After this course of treatment, the treated skin will have reduced redness and reduced inflammation.

### Example 3

A human suffering from acne is treated with SOLARAZE. Approximately 1-2 grams is rubbed into the skin to be treated 1-2 times a day for a period of 90 days. After this course of treatment, the treated skin will have reduced redness and reduced inflammation.

While the invention has been described in detail and with reference to specific examples thereof, it will be apparent to one skilled in the art that various changes and modifications can be made therein without departing from the spirit and scope thereof.

1. A method of treating photodamage-induced wrinkles, said method comprising:
providing a composition consisting essentially of 1-3 wt. % diclofenac or a salt thereof, 0.5-1.5 wt. % benzyl alcohol, 10-30 wt. % PEG monomethyl ether, 0.1-3.0 wt. % sodium hyaluronate, and 70-80 wt. % water; and
applying the composition to skin of a person in need of said treating in an amount effective to treat the photodamage-induced wrinkles.

2. The method of claim 1, wherein about 1-2 grams of the composition is rubbed into the skin 1-2 times a day for at least 90 consecutive days.

3-8. (canceled)

9. The method of claim 1, wherein the composition consists essentially of about 3 wt. % diclofenac sodium, about 1.0 wt. % benzyl alcohol, about 20 wt. % PEG monomethyl ether, about 2.5 wt. % sodium hyaluronate and about 73.5 wt. % water.

10. A method of reducing a severity of photodamage-induced wrinkles, said method comprising:
providing a composition consisting essentially of about 3 wt. % diclofenac sodium, about 1.0 wt. % benzyl alcohol, about 20 wt. % PEG monomethyl ether, about 2.5 wt. % sodium hyaluronate and about 73.5 wt. % water; and
repeatedly applying the composition to skin of a person in need of said treating in an amount effective to reduce the severity of the photodamage-induced wrinkles on the skin.

11. The method of claim 10, wherein about 1-2 grams of the composition is rubbed into the skin 1-2 times a day for at least 180 consecutive days.

12-15. (canceled)

16. The method of claim 1, wherein about 0.5-5 grams of the composition is rubbed into the skin 1-2 times a day.

17. The method of claim 10 wherein about 0.5-5 grams of the composition is rubbed into the skin 1-2 times a day.

18. A method of reducing a severity of photodamage-induced wrinkles, said method comprising:
providing a composition consisting essentially of 1-3 wt. % diclofenac or a salt thereof, 0.5-1.5 wt. % benzyl alcohol, 10-30 wt. % PEG monomethyl ether, 0.1-3.0 wt. % sodium hyaluronate, and 70-80 wt. % water; and
repeatedly applying the composition to skin of a person in need of said treating in an amount effective to reduce the severity of the photodamage-induced wrinkles on the skin.

19. The method of claim 18, wherein about 0.5-5 grams of the composition is rubbed into the skin 1-2 times a day.

20. The method of claim 18, wherein about 1-2 grams of the composition is rubbed into the skin 1-2 times a day for at least 90 consecutive days.

21. The method of claim 18, wherein about 1-2 grams of the composition is rubbed into the skin 1-2 times a day for at least 180 consecutive days.

22. The method of claim 1, wherein the composition consists of 1-3 wt. % diclofenac or a salt thereof, 0.5-1.5 wt. % benzyl alcohol, 10-30 wt. % PEG monomethyl ether, 0.1-3.0 wt. % sodium hyaluronate, and 70-80 wt. % water.

23. The method of claim 10, wherein the composition consists of 1-3 wt. % diclofenac or a salt thereof, 0.5-1.5 wt. % benzyl alcohol, 10-30 wt. % PEG monomethyl ether, 0.1-3.0 wt. % sodium hyaluronate, and 70-80 wt. % water.

24. The method of claim 18, wherein the composition consists of 1-3 wt. % diclofenac or a salt thereof, 0.5-1.5 wt. % benzyl alcohol, 10-30 wt. % PEG monomethyl ether, 0.1-3.0 wt. % sodium hyaluronate, and 70-80 wt. % water.

25. The method of claim 1, wherein the composition consists of about 3 wt. % diclofenac sodium, about 1.0 wt. % benzyl alcohol, about 20 wt. % PEG monomethyl ether, about 2.5 wt. % sodium hyaluronate and about 73.5 wt. % water.

26. The method of claim 10, wherein the composition consists of about 3 wt. % diclofenac sodium, about 1.0 wt. % benzyl alcohol, about 20 wt. % PEG monomethyl ether, about 2.5 wt. % sodium hyaluronate and about 73.5 wt. % water.

27. The method of claim 18, wherein the composition consists of about 3 wt. % diclofenac sodium, about 1.0 wt. % benzyl alcohol, about 20 wt. % PEG monomethyl ether, about 2.5 wt. % sodium hyaluronate and about 73.5 wt. % water.
